(19) United States
(12) Patent Application Publication
(10) Pub. No.: US 2006/0200039 A1
(43) Pub. Date: Sep. 7, 2006

(54) Impedance based sensor for monitoring leakage in AAA stent graft

(75) Inventors: Brian P. Brockway, Shoreview, MN (US); Perry A. Mills, Arden Hills, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Transoma Medical, Inc., St. Paul, MN (US)

(21) Appl. No.: 11/357,721

(22) Filed: Feb. 16, 2006

Related U.S. Application Data
(60) Provisional application No. 60/653,356, filed on Feb. 16, 2005.

(51) Int. Cl. A61B 5/05

(52) U.S. Cl. 600/547

(57) ABSTRACT

Embodiment of the invention provide a technique for detecting endoleakage of an abdominal aortic aneurism (AAA) stent graft on a relatively frequent basis at home or the clinic without the safety risks and/or costs associated with current approaches. In one embodiment, an apparatus for detecting leakage in an AAA graft comprises: an electrode array having a plurality of electrodes distributed over and coupled with a surface of the AAA graft; and an electrical circuit configured to generate a stimulus voltage or current to be applied between sets of the plurality of electrodes of the electrode array and measure an impedance between the sets of the plurality of electrodes. The sets of electrodes for measuring the impedance are same as or different from the sets of electrodes for applying the stimulus voltage or current. A leakage is detected by a decrease in the impedance measured by the electrical circuit.

### CROSS-REFERENCES TO RELATED APPLICATIONS

This application claims the benefit of U.S. Provisional Patent Application No. 60/653,356, filed Feb. 16, 2005, the entire disclosure of which is incorporated herein by reference.

### BACKGROUND OF THE INVENTION

The application relates generally to leakage detection and, more particularly, to the detection of leakage of blood into an aneurismal sac following placement of a graft to treat an abdominal aortic aneurism (AAA).

Leakage of blood following placement of AAA grafts, especially AAA stent grafts, is a common problem. Currently patients must be examined at regular intervals following graft placement using imaging (e.g., ultrasound or CAT) to assess whether the graft is leaking. Leakage can occur from four different causes: (1) leakage due to a poor seal between the graft and the vessel wall, (2) blood flow into the aneurismal sac through collateral circulation, (3) leakage due to mechanical failure of the graft system, and (4) leakage through the graft wall. Leakage can cause pressure to build up within the aneurismal sac resulting in bursting of the sac, loss of blood, and possibly death.

CT (computer tomography) scans, the most common approach to evaluating leakage, have an associated risk, and are expensive. Because of the risk and the cost involved, physicians are reluctant to perform a CT scan more than about once every 6 weeks. The U.S. Food and Drug Administration (FDA) estimates that a CT examination with an effective dose of 10 millisieverts (mSv), for example, 1 CT examination of the abdomen, may be associated with an increase in the possibility of fatal cancer of approximately 1 chance in 2000. See http://www.fda.gov/cdrh/ct/risks.html.

### BRIEF SUMMARY OF THE INVENTION

Embodiments of the invention provide a technique for detecting leakage of blood into an aneurismal sac on a relatively frequent basis at home or in the clinic without the safety risks and/or costs associated with current approaches. Such a technique is beneficial to the patient and reduces cost. Detection of leakage at an early stage allows intervention before the safety of the patient is significantly compromised. In specific embodiments, an implanted device measures changes in impedance within the AAA stent graft and particularly within the vicinity of the aneurismal sac indicating that potentially dangerous leakage may be occurring and may result in bursting of the aneurismal sac.

In accordance with an aspect of the present invention, an apparatus for detecting leakage in an abdominal aortic aneurism (AAA) graft comprises: an electrode array having a plurality of electrodes distributed over and coupled with a surface of the AAA graft; and an electrical circuit configured to generate a stimulus voltage or current to be applied between sets of the plurality of electrodes of the electrode array and measure an impedance between sets of the plurality of electrodes. The sets of electrodes for measuring the impedance are same as or different from the sets of electrodes for applying the stimulus voltage or current. A leakage is detected by a decrease in the impedance measured by the electrical circuit.

In some embodiments, the electrode array includes two sets of electrodes, each set of electrodes being connected in parallel, and the electrical circuit is configured to apply the stimulus voltage or current between the two sets of electrodes and measure the impedance between the two sets of electrodes. The electrode array may include at least one linear array of electrodes. In this embodiment, the stimulus must connect to both sets of electrodes to create a circuit. The linear array may include alternating pairs of electrodes forming two sets of alternating electrodes. The electrode array may be wrapped around the surface of the AAA graft in a spiral manner. A plurality of linear arrays of electrodes may be distributed around the surface of the AAA graft. The electrical circuit is configured to measure the impedance of each array of electrodes independently to detect any local decrease in the impedance. Alternatively, the sets of electrodes in the plurality of linear arrays are connected in parallel, and the electrical circuit is configured to obtain a composite measurement of the impedance of the sets of electrodes in the plurality of linear arrays connected in parallel. The stimulus voltage or current may be a pulse stimulus or a sinusoidal stimulus. The stimulus voltage or current may be a sinusoidal stimulus having a single frequency. The stimulus voltage or current may be a sinusoidal stimulus having two or more different, alternately applied frequencies. The electrical circuit is configured to isolate reactive and resistive components of the measured impedance and detect leakage based on at least one of the isolated reactive and resistive components.

In specific embodiments, the AAA graft is a stent graft having a plurality of conductive struts, and the electrodes of the electrode array include at least some of the conductive struts. The stent graft may include a plurality of conductive rings spaced from each other, and two sets of electrodes are formed by alternating conductive rings. The two sets of alternating conductive rings are connected in parallel, and the electrical circuit is configured to apply the stimulus voltage or current between the two sets of alternating conductive rings and measure the impedance between the two sets of alternating conductive rings. In this embodiment, the stimulus must connect to both sets of electrodes to create a circuit.

In some embodiments, a control unit is configured to control impedance measurement by the electrical circuit including a timing of the impedance measurement. A telemetry monitoring device having the electrode array, the electrical circuit, the control unit, a memory, at least one antenna, a transmitter, and a receiver is configured to be implanted into a body of a patient. A receiver/activator is disposed remotely from the control unit, and configured to communicate with the control unit via the transmitter and the receiver. A monitoring station is disposed remotely from and in communication with the receiver/activator to receive and process impedance data from the receiver/activator. A base station communicates wirelessly with the receiver/activator to transmit data therebetween and communicates with the monitoring station via a communications link.

In specific embodiments, the telemetry monitoring device includes a battery. The telemetry monitoring device includes a storage element configured to store energy which is inductively coupled from the receiver/activator. The antenna comprises a conductive element coupled with the surface of the AAA graft. The AAA graft is a stent graft having a plurality of conductive struts, and the antenna includes at least one of the conductive struts.

In accordance with another aspect of the present invention, a method of detecting leakage in an abdominal aortic aneurism (AAA) graft comprises: providing an electrode array having a plurality of electrodes distributed over and coupled with a surface of the AAA graft; and applying a stimulus voltage or current between sets of the plurality of electrodes of the electrode array and measure an impedance between the sets of the plurality of electrodes. The sets of electrodes for measuring the impedance are same as or different from the sets of electrodes for applying the stimulus voltage or current. A leakage is detected by a decrease in the measured impedance.

In some embodiments, measuring the impedance comprises measuring the impedance of each array of electrodes independently to detect any local decrease in the impedance. Alternatively, the sets of electrodes in the plurality of linear arrays are connected in parallel, and measuring the impedance comprises obtaining a composite measurement of the impedance of the sets of electrodes in the plurality of linear arrays connected in parallel. The method may further include isolating reactive and resistive components of the measured impedance and detecting leakage based on at least one of the isolated reactive and resistive components.

In specific embodiments, the method further comprises implanting into a body of a patient a telemetry monitoring device having the electrode array, the electrical circuit, the control unit, a memory, at least one antenna, a transmitter, and a receiver. The method may further comprise communicating wirelessly with the control unit via the transmitter and the receiver. The telemetry monitoring device includes a storage element, and the method includes inductively coupling energy from a receiver/activator to the telemetry monitoring device and storing the energy in the storage element.

### BRIEF DESCRIPTION OF THE DRAWINGS

FIG. 1 is a simplified schematic diagram showing a telemetry device connected electrically to an electrode array located on the outer surface of a graft for leakage monitoring according to an embodiment of the present invention.

FIG. 2 is a simplified schematic diagram of a longitudinal electrode array in which impedance is measured between alternating pairs of electrodes according to one embodiment of the invention.

FIG. 3 is a simplified schematic diagram of a longitudinal electrode array in which the electrodes are grouped into multiple sets of 4 according to another embodiment of the invention.

FIG. 4 shows a plot of the stimulus voltage applied to one electrode set relative to the other electrode set in the electrode array of FIG. 2, and a plot of the resulting current.

FIG. 5 shows an RC circuit model for the impedance between electrodes.

FIG. 6 is a simplified schematic diagram of a longitudinal electrode array in which groups of the electrodes are used to selectively measure impedance in localized areas according to another embodiment of the invention.

FIG. 7 is a simplified schematic diagram showing an arrangement of multiple arrays of electrodes for sensing impedance according to another embodiment

FIG. 8 is a simplified schematic diagram of an electrode arrangement in which the struts of the stent graft are used as electrodes for sensing impedance according to another embodiment of the invention.

FIG. 9 is system for monitoring stent graft leakage according to an embodiment of the invention.

FIG. 10 is a block diagram of a telemetry monitoring device according to an embodiment of the invention.

### DETAILED DESCRIPTION OF THE INVENTION

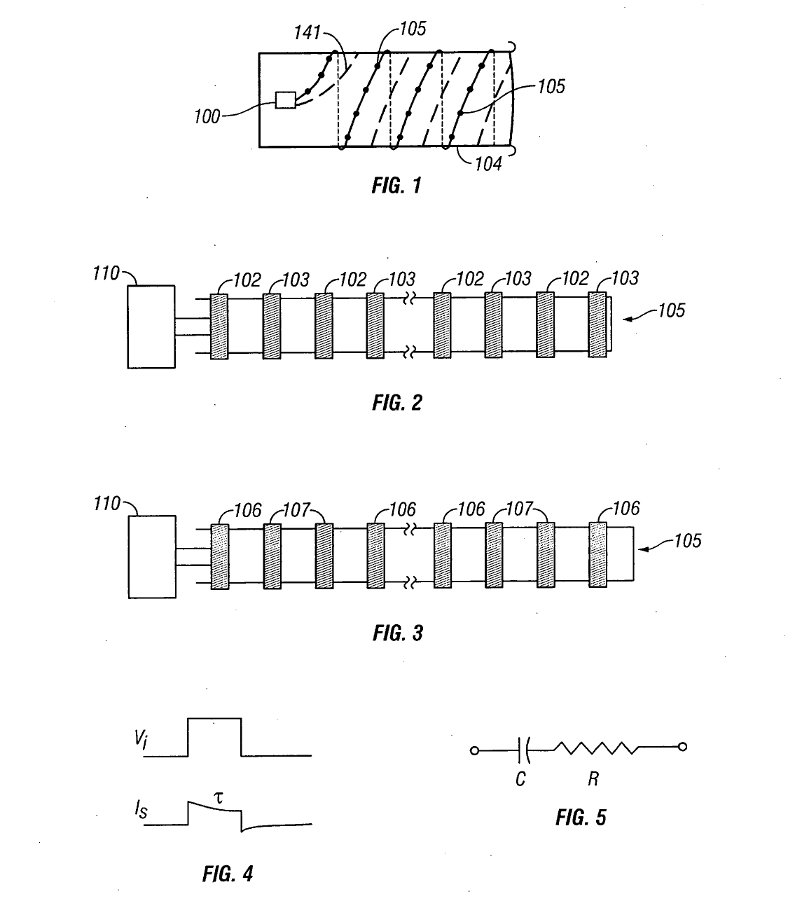

As shown in FIG. 1, a telemetry device 100, which is configured to be attached to an AAA graft 104, is connected electrically to an electrode array 105 located on the outer surface of the graft 104. The telemetry device is a telemetry monitoring device (TMD) that controls impedance measurement, collects the impedance measurement data, stores the data within the TMD, and transmits the impedance measurement data to a receiver that receives the data outside the body and displays or reports the information to a caregiver.

In one embodiment, the electrode array 105 includes a longitudinal array in which impedance is measured between alternating pairs of electrodes. FIG. 2 shows such an array having two sets of electrodes (102 and 103). Electrodes in the set 102 are electrically connected in parallel, as are electrodes in the set 103. The electrode sets 102 and 103 alternate in location along the length of the array 105. An electrical circuit or impedance measuring circuit 110 generates a stimulus voltage such as a voltage pulse and measures the impedance between the electrode sets 102 and 103 along the length of the array 105. If a leakage occurs at any point along the electrode array 105, the impedance between the electrode sets 102, 103 will decrease.

With this type of array 105, where two sets of electrodes 102, 103 are used, it may be advantageous to measure the reactive and resistive components of impedance independently using the circuit 110. This can be accomplished with low power by applying a voltage pulse on the electrode set 102 relative to the electrode set 103. As seen in FIG. 4, Vi is the voltage on the electrode set 102 relative to the electrode set 103. The maximum value of the current Is indicates the resistive component of the impedance, while the time constant of the fall-off in the current Is following termination of the stimulus voltage is dependent upon both the reactive and resistive components of the impedance. The reactive component of impedance can be isolated by mathematical modeling of the system as an RC circuit as illustrated in FIG. 5. Once the resistive component has been identified by measurement of the current Is, the reactive (capacitive) component can be calculated using conventional circuit analysis techniques. Likewise, a stimulus current pulse may be used such that the starting voltage across the electrodes indicates the resistive component and the rate of voltage increase indicates the reactive component. Isolating the reactive and resistive components of the impedance may allow the sensor to be more sensitive to detecting the ingress of blood, since the reactive component or the resistive component may be more sensitive to detecting the ingress of blood, depending on the particular set of conditions.

An alternative embodiment of this approach to the linear array, where two sets of electrodes are employed, is to measure impedance by applying a sinusoidal stimulus between the electrode sets 102, 103 and measuring the resulting current with the circuit 110. In yet another embodiment, the electrodes may be driven with a current and the voltage is measured in response.

FIG. 3 shows another embodiment of the electrode array 105. In this embodiment, the electrodes are grouped into multiple sets of 4 that extend along the length of the electrode array 105. The two electrode sets 106, 107 are arranged in multiple sets of 4 (106, 107, 107, 106). The electrode sets 106 are driven by a constant amplitude sinusoidal voltage generated by the circuit 110. The circuit 110 also measures the resulting current induced in the electrode sets 107. The current induced in the electrode sets 107 is indicative of the impedance in the tissue and blood in the vicinity of the electrodes. If the graft leaks and fresh blood enters the area at or near the vicinity of the electrode array 105, the impedance measured by the circuit 110 will drop. In one specific embodiment, the frequency of the sinusoidal voltage is in the range of about 25 kHz to about 150 kHz. In an alternate embodiment, the reactive and resistive components of impedance are measured by stimulating the electrodes with a spectrum of frequencies. This can be done by sweeping the frequency or by applying two or more discrete frequencies in sequence repeatedly. For example, the electrodes are stimulated with a 100 kHz frequency for 1 second to measure the impedance, and then at 50 kHz for 1 second. The impedance at each frequency would be recorded and telemetered as independent values. Signal processing implemented external to the TMD 100 will be employed to compute the reactive and resistive components. Alternatively, the resistive and reactive components may be derived using analog or digital circuitry of the TMD 100. The electrode configuration of FIG. 3 is amenable to pulsatile stimulus, although there may be less reason to separate the reactive and resistive components since the reactive (capacitive) component is oftentimes associated with the electrode/tissue interface.

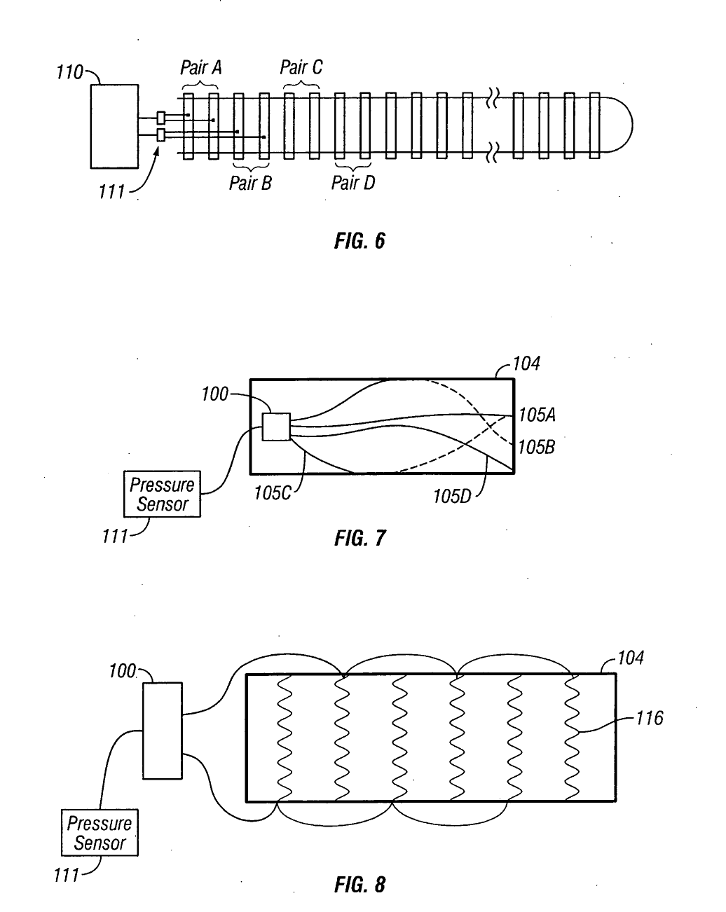

FIG. 6 shows another embodiment of a longitudinal electrode array in which groups of the electrodes are used to selectively measure impedance in localized areas. A plurality of groups of electrodes are connected to the impedance measuring circuit 110 via the impedance measurement front end 111. FIG. 6 shows pairs A, B, C, D, etc., which may be separately connected to the impedance measurement front end 111 to allow the circuit 110 to selectively measure impedance in localized areas of the stent grant surface. This will allow improved sensitivity to ingress of blood in localized areas of the stent graft.

FIG. 7 shows an arrangement of multiple arrays of electrodes according to another embodiment. The use of multiple arrays, 105a, 105b, 105c, 105d allows for thorough sensing coverage of the surface of the stent without the need to form a tight spiral of the linear array around the surface. Instead, the multiple linear electrode arrays will meander along the length of the graft 104. This may allow the stent graft 104 to be more tightly compressed for introduction to the vessel through a smaller opening.

In one embodiment where multiple electrode arrays are employed, all electrode arrays are connected in parallel to provide a composite measurement of impedance that is measured by the circuit 110 and stored in the TMD 100 for later transmission to the receiver/activator.

In an alternate embodiment, the circuit 110 measures the impedance of each electrode array independently. The impedance measurements from the multiple arrays are stored and transmitted to the receiver/activator. Subsequently they are processed by a computer system to assess the differences in measurements that may be indicative of leakage. This is based on the assumption that if there is localized leakage, there will be significant differences in impedance between the arrays. This approach may provide a more sensitive measure that is obtained by a composite measure of impedance from all the arrays.

In each of the above embodiments, the linear electrode arrays may be secured to the graft in any of a number of ways including the use of adhesives, stitching to the fabric of the graft, or imbedding the arrays within a channel or pocket formed of the graft material.

FIG. 8 shows another embodiment in which the struts of the stent graft 104 are used as electrodes for sensing impedance. In this embodiment, alternating struts (typically in the form of rings 116), which may be Nitinol struts, are interconnected in a manner similar to that of the electrodes (102, 103) shown in FIG. 2 to form two sets of alternating struts 116 connected in parallel. In a typical stent graft, linear elements of Nitinol are often used to stabilize the struts. In this case, the struts are stabilized by securing them to the underlying fabric, such as Dacron or ePTFE (expanded polytetrafluoroethylene) fabric. If additional stabilization is required, longitudinal members of an insulating material such as HDPE (high density polyethylene) can be used to stabilize the struts in the embodiment of FIG. 8.

In an alternate embodiment, instead of monitoring impedance with blood contacting electrodes, other material properties such as dielectric constant may be monitored with one or more non-contacting probes. In one embodiment, the probe is driven with a voltage or a current at a frequency chosen to maximize the sensitivity to ingress of blood. Dielectric constant would be assessed by measuring the capacitance of the probe to the surrounding tissue. Capacitance would be derived from the relationship of current and voltage delivered to the probe as determined by the formula I=C(dv/dt). The probe may be a dedicated protrusion of the TMD 100 and electrically connected to the TMD 100 or may be one or more insulated struts of the graft that are electrically connected to the TMD 100. Capacitance may be measured from probe to probe or from each probe to a larger common structure such as the struts of the graft. The larger common structure is not required to be insulated from the surrounding tissue. At high frequencies, other material properties such as loss-tangent may be monitored to detect ingress of blood.

In an alternate embodiment, a pressure sensor (see optional pressure sensor 111 in FIGS. 6 and 7) is also placed with the stent in a manner similar to that used in the prior art. The pressure sensor would be electrically connected to the transmitting electronics in TMD 100. Pressure data would be stored and transmitted along with the impedance data and would provide an extra measure of diagnostic information. Because of the need to perform barometric pressure correction on the pressure value obtained from the aneurism, it would be most convenient if the pressure signal were only obtained at the time TMD 100 is interrogated by the activator, since this approach would allow the barometric pressure sensor located in the activator to provide a measurement to subtract from the value obtained from the TMD 100 in real time.

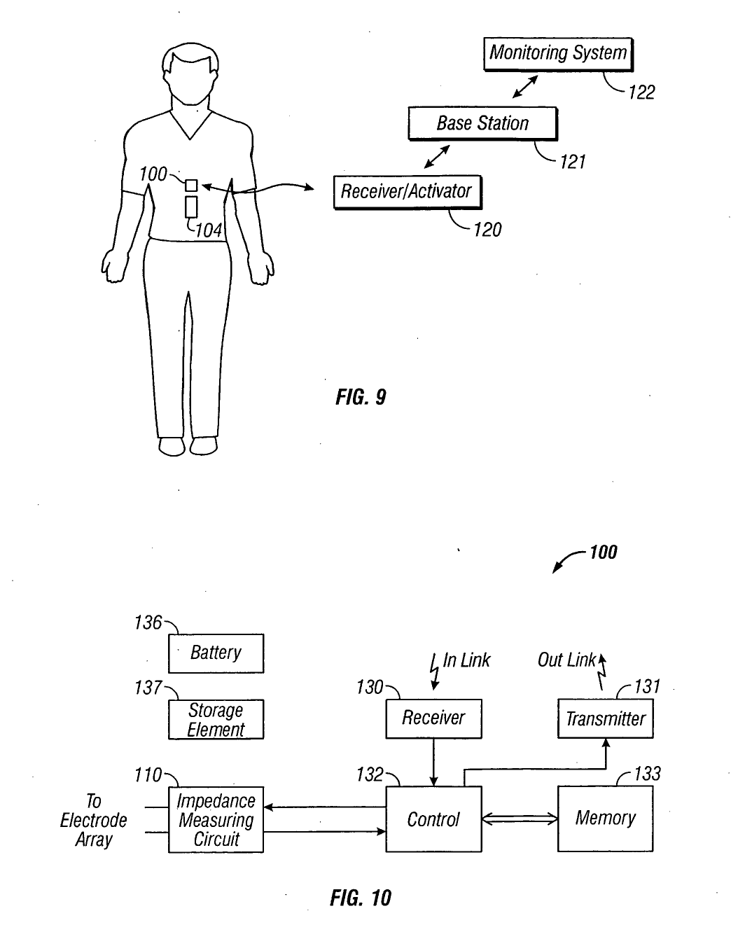

FIG. 9 shows a system for monitoring stent graft leakage which includes the telemetry monitoring device (TMD) 100 and associated electrode array(s) and antenna implanted in the patient along with the stent graft 104. A receiver/activator 120 is used to program certain features of the TMD 100 such as how often it samples impedance and which electrode sets should be used in combination to measure impedance. The receiver/activator 120 also signals the TMD 100 to transmit stored information and receives such stored information from the TMD 100. A base station 121 communicates wirelessly with the receiver/activator 120. Patient data received from the receiver/activator 120 is forwarded by the base station 121 via a communications link to a clinic monitoring system or station 122 in a clinic, hospital, or monitoring center. The communications link may be a cellular link, Internet, or land line. The clinical monitoring system 122 is a computerized system that receives impedance data, analyzes the information to extract possible signatures indicative of leakage, and provides displays and reports to caregivers or monitoring center personnel that allow them to evaluate the presence of leakage in the stent graft 104 being monitored. In cases where the data is read in the physician's office directly with the patient present, the information may be readable directly on the receiver/activator 120 or be transferable directly from the receiver/activator 120 to the clinic monitoring system 122. In one embodiment, the transmitter and receiver share a common antenna, whereas in an alternate embodiment, the transmitter and receiver operate on significantly different frequencies and employ separate antennae.

The clinic monitoring system 122 may include the ability to evaluate long term trends in impedance data sampled from the stent graft. It would be anticipated that following a healing time after placement of the stent graft that in the absence of endoleakage, the impedance would be reasonably stable and would decrease if leakage were to occur. The clinic monitoring system 122 would therefore provide the ability to observe trends in impedance data over weeks or months in order to identify clinically relevant changes that may warrant action on the part of the caregiver.

The TMD 100 may be configured in any suitable manner. One example is illustrated in FIG. 10. A control unit or circuitry 132 controls the timing of impedance measurement by the impedance measuring unit (i.e., the electrical circuit 110). The control unit 132 transfers data and programming information to and from a memory 133. A receiver 130 receives signals from the receiver/activator 120 that instruct the TMD 100 to send stored data or provide new programming information. A transmitter 131 transmits information to the receiver/activator 120 such as stored data or control parameters.

In one embodiment, the TMD 100 includes a battery 136 that powers all circuits including transmitter 131 and receiver 130 circuits. The battery 136 may be non-rechargeable or may be recharged via inductive coupling of energy from the receiver/activator 120 or another external device. The battery 136 may also be recharged via energy derived from the pulsatile blood pressure asserted on the interior of the graft. The pulsatile blood pressure may be converted from mechanical energy to electrical via a piezoelectric or other mechanical-to-electrical converter element extending from the TMD 100. The converter element may be coupled to one or more struts supporting the graft to gather energy from a larger surface area of the graft than that encompassed by the element itself. In another embodiment, the TMD 100 contains a battery that powers all circuits except the transmitter 131 circuit. Power for the transmitter 131 is obtained from inductive coupling of energy from the receiver/activator 120. Power inductively coupled into the TMD 100 is used to charge a storage element 137 that stores sufficient energy to power the transmitter 131 for a sufficient period of time to transmit all information stored in memory.

In another embodiment, the struts and/or complete metallic support of the stent graft are used as an antenna to facilitate telemetering of information into and/or out of the patient's body. In this embodiment, an electrical connection from the TMD 100 will be made to the stent metallic structure, either directly or via a capacitor, to couple radio frequency energy from the stent metallic structure to the transmitter 131 and receiver 130 of the TMD 100.

In yet another embodiment, a longitudinal conductive element will be connected to the TMD 100 for use as an antenna 141 (see FIG. 1). This conductive element 141 may either extend longitudinally along the graft 104 or wind around the graft 104 similar to the linear electrode array 105.

Compared to CT and ultrasound imaging, the present approach offers the ability to evaluate the patients for graft leaks on a regular and relatively frequent basis (e.g., weekly or more frequently) while the patient remains at home. This will promote early detection of a leak and prompt intervention to minimize the consequences. Compared to CT scans, this approach will avoid the risk of radiation exposure.

U.S. Pat. No. 6,840,956 discloses the use of a biosensor attached to the stent graft and disposed within the aneurismal sac for allowing remote monitoring of pressure or other conditions to detect an endoleak or excessive pressure therein. See also U.S. Patent Publication No. 2004/0044393. The entire disclosures of these references are incorporated herein by reference.

Compared to the approach disclosed in U.S. Pat. No. 6,840,956, the present approach for leakage detection will result in a graft that is less sensitive to alignment relative to the location of the aneurism than the use of a pressure sensor or other sensors that provide only an isolated area of sensitivity to changes that occur as a result of leakage. U.S. Pat. No. 6,840,956 employs a pressure telemetry device attached to the graft. In order to achieve a high level of sensitivity and specificity, the pressure sensor must be placed within the aneurismal sac in order to detect useful pressure changes. If the sac only extends over a 45 degree portion of the vessel, for example, the graft must be aligned rotationally such that the pressure sensor falls within the region where the sac is located. Further, the graft must be aligned longitudinally so as to assure that the pressure sensor is within the sac. As compared to U.S. Pat. No. 6,840,956, therefore, the present invention will simplify the task of placing the graft and provide for a more robust approach for the placement of the graft.

Moreover, the present approach will likely provide a greater sensitivity to leakage detection and allow leaks to be detected at a very early stage. This results from the use of a sensing array that is distributed over the surface of the graft. The early stage detection also results from sensing the initial presence of blood without requiring a large enough volume of blood to elevate the pressure in the aneurismal sac.

In addition, the use of this approach will allow trending of information for the assessment of leakage status. In contrast, the use of pressure measurement requires a pressure reference indicative of barometric pressure, against which measurements from the implant are referenced. It is possible for the patient to carry a barometric pressure monitor, but this requires patient compliance and is an inconvenience. Further, use of pressure measurement requires that a reader be used to interrogate pressure. Limitations of the pressure measurement approach, the need for an expensive reader, and the need to use an ultrasonic coupling media to obtain a pressure measurement are complicating factors that will likely limit the clinical use of that technique. Furthermore, changes in posture, intestinal gas, and other abdominal content may produce artifact in the pressure measurement that could lead to false alarms.

The present approach will allow for impedance to be measured at regular intervals (e.g., 4 to 12 times per day) and stored within the measuring device. A receiving or interrogating device placed in the home can receive information from the patient on a daily, weekly, or monthly basis, which may be selectable by the physician or monitoring personnel, and can forward the information to a clinic or service center for evaluation. Trending of patient data in this manner will reduce the possibility of false alarms that may otherwise occur when pressure sensors are used to evaluate leakage, and will likely increase the ability of the clinician to identify leakage at an early stage.

It is to be understood that the above description is intended to be illustrative and not restrictive. Many embodiments will be apparent to those of skill in the art upon reviewing the above description. The scope of the invention should, therefore, be determined not with reference to the above description, but instead should be determined with reference to the appended claims along with their full scope of equivalents.

What is claimed is:

1. An apparatus for detecting leakage in an abdominal aortic aneurism (AAA) graft, the apparatus comprising:
an electrode array having a plurality of electrodes distributed over and coupled with a surface of the AAA graft; and
an electrical circuit configured to generate a stimulus voltage or current to be applied between sets of the plurality of electrodes of the electrode array and measure an impedance between sets of the plurality of electrodes;
wherein the sets of electrodes for measuring the impedance are same as or different from the sets of electrodes for applying the stimulus voltage or current;
wherein a leakage is detected by a decrease in the impedance measured by the electrical circuit.

2. The apparatus of claim 1 wherein the electrode array includes two sets of electrodes, each set of electrodes being connected in parallel, and wherein the electrical circuit is configured to apply the stimulus voltage or current between the two sets of electrodes and measure the impedance between the two sets of electrodes.

3. The apparatus of claim 1 wherein the electrode array includes at least one linear array of electrodes.

4. The apparatus of claim 3 wherein the linear array includes alternating pairs of electrodes forming two sets of alternating electrodes.

5. The apparatus of claim 3 wherein the electrode array is wrapped around the surface of the AAA graft in a spiral manner.

6. The apparatus of claim 3 wherein a plurality of linear arrays of electrodes are distributed around the surface of the AAA graft.

7. The apparatus of claim 6 wherein the electrical circuit is configured to measure the impedance of each array of electrodes independently to detect any local decrease in the impedance.

8. The apparatus of claim 6 wherein the sets of electrodes in the plurality of linear arrays are connected in parallel, and the electrical circuit is configured to obtain a composite measurement of the impedance of the sets of electrodes in the plurality of linear arrays connected in parallel.

9. The apparatus of claim 1 wherein the stimulus voltage or current is a pulse stimulus or a sinusoidal stimulus.

10. The apparatus of claim 9 wherein the stimulus voltage or current is a sinusoidal stimulus having a single frequency.

11. The apparatus of claim 9 wherein the stimulus voltage or current is a sinusoidal stimulus having two or more different, alternately applied frequencies.

12. The apparatus of claim 1 wherein the electrical circuit is configured to isolate reactive and resistive components of the measured impedance and detect leakage based on at least one of the isolated reactive and resistive components.

13. The apparatus of claim 1 wherein the AAA graft is a stent graft having a plurality of conductive struts, and wherein the electrodes of the electrode array include at least some of the conductive struts.

14. The apparatus of claim 13 wherein the stent graft includes a plurality of conductive rings spaced from each other, and wherein two sets of electrodes are formed by alternating conductive rings.

15. The apparatus of claim 14 wherein the two sets of alternating conductive rings are connected in parallel, and wherein the electrical circuit is configured to apply the stimulus voltage or current between the two sets of alternating conductive rings and measure the impedance between the two sets of alternating conductive rings.

16. The apparatus of claim 1 further comprising a control unit configured to control impedance measurement by the electrical circuit including a timing of the impedance measurement.

17. The apparatus of claim 16 further comprising a memory, at least one antenna, a transmitter, and a receiver, wherein a telemetry monitoring device having the electrode array, the electrical circuit, the control unit, the memory, the antenna, the transmitter, and the receiver is configured to be implanted into a body of a patient.

18. The apparatus of claim 17 further comprising a receiver/activator disposed remotely from the control unit, and configured to communicate with the control unit via the transmitter and the receiver.

19. The apparatus of claim 18 further comprising a monitoring station disposed remotely from and in communication with the receiver/activator to receive and process impedance data from the receiver/activator.

20. The apparatus of claim 19 further comprising a base station which communicates wirelessly with the receiver/activator to transmit data therebetween and communicates with the monitoring station via a communications link.

21. The apparatus of claim 17 wherein the telemetry monitoring device includes a battery.

22. The apparatus of claim 21 wherein the telemetry monitoring device includes a storage element configured to store energy which is inductively coupled from the receiver/activator.

23. The apparatus of claim 17 wherein the antenna comprises a conductive element coupled with the surface of the AAA graft.

24. The apparatus of claim 17 wherein the AAA graft is a stent graft having a plurality of conductive struts, and wherein the antenna includes at least one of the conductive struts.

25. The apparatus of claim 1 further comprising a pressure sensor configured to measuring pressure in an aneurismal sac to which the AAA graft is coupled.

26. A method of detecting leakage in an abdominal aortic aneurism (AAA) graft, the method comprising:
providing an electrode array having a plurality of electrodes distributed over and coupled with a surface of the AAA graft; and
applying a stimulus voltage or current between sets of the plurality of electrodes of the electrode array and measure an impedance between sets of the plurality of electrodes;
wherein the sets of electrodes for measuring the impedance are same as or different from the sets of electrodes for applying the stimulus voltage or current;
wherein a leakage is detected by a decrease in the measured impedance.

27. The method of claim 26 wherein the electrode array includes two sets of electrodes, each set of electrodes being connected in parallel, and wherein the stimulus voltage or current is applied between the two sets of electrodes and the impedance is measured between the two sets of electrodes.

28. The method of claim 26 wherein the electrode array includes at least one linear array of electrodes.

29. The method of claim 28 wherein the linear array includes alternating pairs of electrodes forming two sets of alternating electrodes.

30. The method of claim 28 wherein the electrode array is wrapped around the surface of the AAA graft in a spiral manner.

31. The method of claim 28 wherein a plurality of linear arrays of electrodes are distributed around the surface of the AAA graft.

32. The method of claim 31 wherein measuring the impedance comprises measuring the impedance of each array of electrodes independently to detect any local decrease in the impedance.

33. The method of claim 31 wherein the sets of electrodes in the plurality of linear arrays are connected in parallel, and wherein measuring the impedance comprises obtaining a composite measurement of the impedance of the sets of electrodes in the plurality of linear arrays connected in parallel.

34. The method of claim 26 wherein the stimulus voltage or current is a pulse stimulus or a sinusoidal stimulus.

35. The method of claim 34 wherein the stimulus voltage or current is a sinusoidal stimulus having a single frequency.

36. The method of claim 34 wherein the stimulus voltage or current is a sinusoidal stimulus having two or more different, alternately applied frequencies.

37. The method of claim 26 further comprising isolating reactive and resistive components of the measured impedance and detecting leakage based on at least one of the isolated reactive and resistive components.

38. The method of claim 26 wherein the AAA graft is a stent graft having a plurality of conductive struts, and wherein the electrodes of the electrode array include at least some of the conductive struts.

39. The method of claim 38 wherein the stent graft includes a plurality of conductive rings spaced from each other, and wherein two sets of electrodes are formed by alternating conductive rings.

40. The method of claim 39 wherein the two sets of alternating conductive rings are connected in parallel, and wherein the stimulus voltage or current is applied between the two sets of alternating conductive rings and the impedance is measured between the two sets of alternating conductive rings.

41. The method of claim 28 further comprising providing a control unit to control impedance measurement by the electrical circuit including a timing of the impedance measurement.

42. The method of claim 41 further comprising implanting into a body of a patient a telemetry monitoring device having the electrode array, the electrical circuit, the control unit, a memory, at least one antenna, a transmitter, and a receiver.

43. The method of claim 42 further comprising communicating wirelessly with the control unit via the transmitter and the receiver.

44. The method of claim 42 wherein the telemetry monitoring device includes a battery.

45. The method of claim 42 wherein the telemetry monitoring device includes a storage element, and further comprising inductively coupling energy from a receiver/activator to the telemetry monitoring device and storing the energy in the storage element.

46. The method of claim 42 wherein the antenna comprises a conductive element coupled with the surface of the AAA graft.

47. The method of claim 42 wherein the AAA graft is a stent graft having a plurality of conductive struts, and wherein the antenna includes at least one of the conductive struts.

48. The method of claim 26 further comprising measuring pressure in an aneurismal sac to which the AAA graft is coupled.
